(19) United States
(12) Patent Application Publication
(10) Pub. No.: US 2007/0125376 A1
(43) Pub. Date: Jun. 7, 2007

(54) EVAPORATOR, ARTIFICIAL RESPIRATION APPARATUS AND EVAPORATION PROCESS

(76) Inventor: Jurgen Reinstadtler, Wurzburg (DE)

Correspondence Address: KING & SCHICKLI, PLLC, 247 NORTH BROADWAY, LEXINGTON, KY, 40507, US

(21) Appl. No.: 11/671,145

(22) Filed: Feb. 5, 2007

Related U.S. Application Data
(63) Continuation of application No. PCT/DE05/01352, filed on Aug. 1, 2005.

(30) Foreign Application Priority Data
Aug. 4, 2004 (DE) 10 2004 037 823.1

(51) Int. Cl. A61M 16/00; F23D 11/00

(52) U.S. Cl. 128/203.26; 128/204.17; 128/203.17; 128/203.27

(57) ABSTRACT

This invention relates to an evaporator which comprises a casing for receiving a liquid, and a heater. The heater comprises a plurality of heating elements which can be heated individually. This invention moreover relates to respirators comprising such evaporators, and to evaporation methods.

### CROSS REFERENCE TO RELATED CO-PENDING APPLICATIONS

This application is a continuation of international application number PCT/DE2005/001352 (publication number. WO 2006/012878 A1) filed on Aug. 1, 2005 and entitled EVAPORATOR, ARTIFICIAL RESPIRATION APPARATUS AND EVAPORATION PROCESS and claims the benefit of the above-mentioned international application and the corresponding German national patent application number 10 2004 037 823.1 filed on Aug. 41, 2004 and entitled VERDAMPFER, BEATMUNGSGERÄT SOWIE VERDAMPFUNGSVERFAHREN the contents of which are expressly incorporated herein by reference.

### FIELD OF THE INVENTION

The invention relates to evaporators, and respirators comprising such evaporators. The invention further relates to an evaporation method.

### BACKGROUND OF THE INVENTION

Respirators are employed, inter alia, in intensive care medicine for the mechanical artificial respiration in all forms of the oxygen deficiency state. To prevent the mucous membranes from desiccating humidifiers are additionally used, which may be integrated in a respirator. Especially in the intensive care medicine high demands are made on the sterilization also of the humidifiers.

One evaporator for the respirators used in intensive care medicine is known from DE 198 08 590 A1. This so-called respiratory humidifier comprises a peristaltic pump as dosing means and an electrically heated evaporator. The peristaltic pump delivers a required quantity of water from a commercial water bag so as to obtain a predetermined relative respiratory gas humidity at a predetermined respiratory gas temperature. The evaporator provides water at a temperature of more than 134° C. which heats the respiratory gas to the predetermined respiratory gas temperature when mixed with the respiratory gas to be humidified. Preferably, a thermal insulation is provided between the outlet side of the evaporator and a respiratory gas channel so as to avoid, if possible, a heating of the respiratory gas channel by the respiratory humidifier also without the supply and evaporation of water. The outlet opening of the evaporator preferably projects into the respiratory gas channel. The high heating temperature results from the desire to destroy germs possibly existing in the water. According to the hygiene regulations for steam sterilization a sufficient reduction of germs is achieved if the germs are exposed to a temperature of 134° C. for three minutes.

In the broader sense respirators also include so-called CPAP-apparatus which serve the treatment of apneas during the sleep. To this end, the CPAP (continuous positive airway pressure) therapy was developed, which is described in Chest. Volume No. 110, pages 1077-1088, October 1996 and in Sleep, Volume No. 19, pages 184-188. A CPAP-apparatus generates a positive airway pressure up to approximately 30 mbar by means of a compressor or turbine and administers the same, preferably via a humidifier, via a tube and via a nose mask, to the respiratory tract of the patient. This positive airway pressure is to ensure that the upper respiratory tract remains fully opened during the whole night, so that no apneas will occur (DE 198 49 571 A1). A humidifier used in conjunction with said CPAP-apparatus prevents the patient's mucous membranes from desiccating.

A respiratory gas humidifier for CPAP-apparatus is described in DE 199 36 499 A1. The humidifier comprises a refill unit formed of a tub element and a pot part coupled therewith, which can be removed from a mountable casing. The tub element and the pot part are imperviously connected with each other. In conjunction with a partition wall a store room for a liquid is formed in said pot part, which contains the major part of the water reserve provided for humidifying the respiratory gas. A separate humidifying area is formed in the tub element disposed underneath the pot part, which merely contains a small portion of the water reserve. The height of the water in the tub element is kept at a predetermined level by a dosing device. In the course of the gradual evaporation of the water located in the tub element water from the liquid store room is successively or continuously refilled. Via a respiratory gas inlet opening the respiratory gas is blown through the upper portion of the tub element to a respiratory gas outlet opening. The bottom area of the tub element is heated by a heating device. For increasing the thermal transmission, the bottom area of the tub element is made of a material having a high thermal conductivity, e.g. Metal.

A humidifier for respirators similar to the one described in DE 199 36 499 A1 is described in DE 200 10 553 U1. In the humidifier according to DE 200 10 553 U1 the air is also passed over the surface of a heatable water reservoir. A water tank, which is substantially integral, is used instead of the refill unit formed of a tub element and a pot part. The water tank has a filling hole which is closed by a cap during the operation.

The humidifier known from DE 101 63 800 A1 comprises a storage tank and a regulating reservoir. A regulating valve operates as floater and closes the opening between the regulating reservoir and the storage tank if the liquid level in the regulating reservoir is high enough. The regulating reservoir is connected to a heating channel having a heating zone for the evaporation of water.

The evaporators described in DE 101 51 397 C1 comprise a storage tank fixed in a casing. The opening of the storage tank faces downwardly during the humidifying operation. Located next to the storage tank is a humidifying area with a gas inlet and a gas outlet. Through a passage in a web touched by the lower edge of the storage tank, or through a notch in the lower edge of the storage tank itself, liquid flows from the storage tank into the humidifying area and covers the bottom thereof with a liquid layer of a specified thickness. This is also called “bird bath principle”. A heating element is arranged underneath the liquid layer.

The German Utility Model 20 2004 004 115.4 also describes an evaporator operating according to the bird bath principle. This evaporator has the particular advantage that it can be filled with water through the air inlet or outlet. This is achieved by a double-walled storage tank assembly, whereby the inner wall is referred to as diverter and separates the store room with the water reserve from the humidifying area. The bottom of the humidifying area is heatable and is covered with a thin water film.

It is desirable to provide a fast evaporator, corresponding respirators as well as an evaporation method.

### SUMMARY OF THE INVENTION

According to an embodiment of the invention an evaporator is provided which comprises a casing for receiving a liquid and a heater. The heater comprises a plurality of heating elements which can be heated individually.

According to another embodiment of the invention a respirator comprises a compressor for delivering gas, an evaporator, a respiratory tube, a temperature sensor and a controller. The inlet of the evaporator is connected to the compressor. Gas is supplied to the evaporator via the connection. On the respirator's side, the end of a respiratory tube is connected to the outlet of the evaporator. The respiratory tube comprises a patient's-side end. The temperature sensor thermally contacts the respiratory tube. The signal of the temperature sensor is provided to the controller which controls the temperature of the individual heating elements such that there is no water condensation in the respiratory tube. The evaporator comprises a casing for receiving a liquid and a heater. The heater comprises a plurality of heating elements which can be heated individually. The casing has two connections, namely an inlet and an outlet, wherein the inlet serves to supply gas and the outlet serves to discharge gas, and wherein the discharged gas is accumulated with one of liquid molecules and atoms.

According to a further embodiment of the invention a respirator is provided which comprises a compressor, an evaporator, a flow sensor and a controller. The compressor delivers gas. A flow sensor determines the flow of the delivered gas. The signal of the flow sensor is provided to the controller which determines inspiration and expiration phases from this signal and heats the heating elements only during an inspiration phase. The evaporator comprises a casing for receiving a liquid and a heater. The heater comprises a plurality of heating elements which can be heated individually. The casing has two connections, namely an inlet and an outlet, wherein the inlet serves to supply gas and the outlet serves to discharge gas, and wherein the discharged gas is accumulated with one of liquid molecules and atoms.

According to yet a further embodiment of the invention an evaporation method is provided. A liquid is heated, wherein a portion of one of the molecules and atoms in the liquid evaporates. A heating element is heated by a heater which comprises a plurality of heating elements. The liquid is in thermal contact with the heated heating element.

In particular, the fastness is achieved by dividing the heater into smaller heating elements.

Due to the small inertia of the evaporator it is advantageously possible to humidify breathing air merely during the inspiration phases, but not during the expiration phases.

A thin liquid film contributes to the smaller inertia of the evaporator.

On correspondingly selecting the bottom material the same is deformed reversibly if a heating element is heated. This deformation counteracts the furring, facilitates the cleaning and hygiene regulations are complied with (more easily).

By means of strip-shaped conductor or metal paths above and underneath a resistive layer, said paths above the resistive layer extending perpendicularly to the paths underneath the resistive layer when viewed from a normal to the resistive layer, the resistive layer can simply be subdivided into a plurality of heating elements.

A thin liquid film above a heating element can be evaporated completely if the thermal energy exceeds a certain threshold. Above this threshold the breathing air can be heated independently of the humidification. Another advantage of the complete evaporation of the liquid film above a heating element is that the absolute humidification of the air or, more generally, the evaporated amount of liquid, becomes independent of the velocity of the following flow of liquid, in particular of the viscosity of the liquid.

The higher the heating power, the faster evaporates the liquid film in an explosion-like manner. The evaporated liquid virtually “shoots” into the gas stream thereby ensuring a better intermixing of gas and liquid molecules or atoms. In other words, a more uniform distribution of the liquid molecules or atoms is obtained by this.

If the temperature of the respiratory tube is considered in the humidification a condensation of water in the respiratory tube can be prevented. The inspiration of water is unpleasant for the patient. Moreover, moist spots in the respiratory tube may serve as breeding-grounds for germs. It is noted in this respect that the temperature in respirators for intensive care medicine is higher than the ambient temperature (compare DE 198 08 590 A1). Therefore, the temperature in the respiratory tube falls from the respirator toward the patient.

Deactivating the humidification during the expiration has a similar effect. In addition, energy is saved.

### BRIEF DESCRIPTION OF THE DRAWINGS

Preferred embodiments of the invention will hereinafter be explained in more detail with reference to the enclosed drawings, wherein like numerals represent like parts. In the drawings:

FIG. 1 shows a vertical section through an evaporator according to the invention;

FIG. 2 shows a horizontal section through the evaporator according to the invention;

FIG. 3 shows a bottom view of the bottom of the evaporator according to the invention; and

FIG. 4 shows a respirator comprising an evaporator according to the invention;

### DETAILED DESCRIPTION OF THE INVENTION

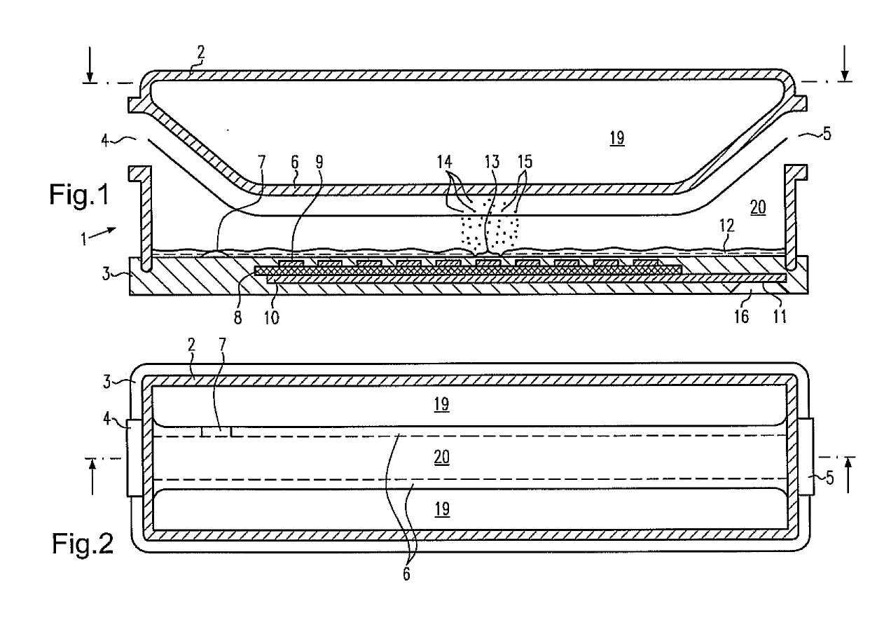

FIG. 1 shows a lateral section through an evaporator 1 according to the invention. The evaporator comprises a storage tank assembly 2 as well as a bottom 3. The storage tank assembly 2 forms an inlet 4, an outlet 5 and a tunnel 6. The tunnel 6 corresponds to the diverter 3 of the German Utility Model 20 2004 004 115.4 and may be formed like the diverter so as to allow the filling of the store room 19 with water via inlet 4 or outlet 5. For the invention this is of minor significance, however.

The evaporator 1 works like the evaporators of some cited documents in accordance with the bird bath principle. In the operating position (FIG. 1) water flows through recess 7 from the store room 19 into the humidifying area 20 until the water film has reached a predetermined thickness thereby closing recess 7. If the thickness of the water film 12 decreases as result of the evaporation, the recess 7 is cleared again so that water may follow from the store room 19. By this, the thickness of the water film 12 is kept largely constant regardless of the liquid level in the store room 19.

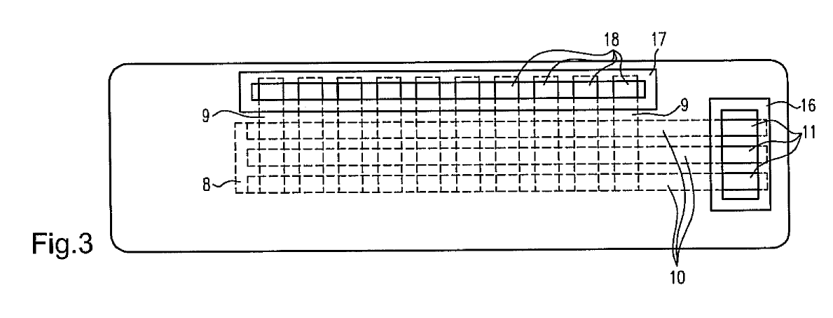

In bottom 3 a heater is provided, which is formed of a plurality of heating elements or is subdivided into a plurality of heating elements. In the embodiment shown in the figures the heater is made of a resistive layer 8 above which upper metal strips 9 and underneath of which lower metal strips 10 are arranged. Viewed from above or below, i.e. From a normal to the plane defined by the resistive layer 8, the upper metal strips 9 appear to run at right angles with respect to the lower metal strips 10 (FIG. 3). At the position where an upper metal strip 9 and a lower metal strip 10 appear to overlap when viewed from below, a heating element is created. If a voltage is applied between the upper and lower metal strips 9, 10 the resultant current flows through the resistive layer 8 substantially in the overlapping region and heats the overlapping region.

Instead of metal strips 9 and 10 strips made of any optional other material may be used, the specific conductivity of which is sufficiently high with respect to the specific conductivity of the resistive layer 8.

In FIG. 1 a non-equilibrium state above the heated heating element 13 is shown. By strongly heating the heating element 13 the water film above heating element 13 evaporated suddenly. The residual water film had not yet had enough time to replace the evaporated water above heating element 13. Above heating element 13 in the humidifying area 20 steam drops 14 and lime particles 15 are illustrated, which are formed by the explosion-like evaporation of the water film. By the explosion-like evaporation the lime dissolved in the water can practically not settle down on bottom 3. In addition, the bottom material, especially its thermal expansion coefficient, can be chosen such that the bottom in the area of the heating element is reversibly deformed when the heating element is temporarily heated, so that the scale chips off.

FIG. 2 shows a horizontal section through an evaporator 1. The recess 7 may be located in the center of evaporator 1 as to render the evaporator more insensitive to inclination.

FIG. 3 shows a bottom view of bottom 3. The upper and lower metal strips 9 and 10 as well as the resistive layer 8 are plotted in a broken manner. Merely in the recess 16 are the lower metal strips 10 exposed and form contact surfaces 11. Similarly, the upper metal strips 9 are exposed in the area of recess 17 and form contact surfaces 18. The contact surfaces 11 and 18 may be gold-plated or coated with any other precious metal to ensure a resistance to corrosion.

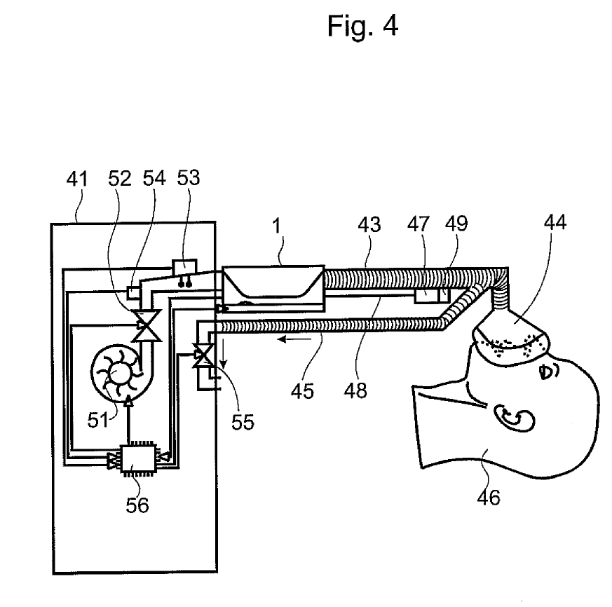

FIG. 4 shows a respirator 41 together with an evaporator 1 according to the invention which acts as humidifier. The respirator 41 comprises a compressor 51 which is also referred to as turbine, ventilator, compressor or blower. During the inspiration phases the air delivered by the compressor 51 flows through valve 52. During the expiration valve 52 is closed to prevent rebreathing and the related contamination of the compressor 51 and sound-absorbing foams preceding the same in the flow direction with causative organisms exhaled by the patient 46. Behind valve 52 the delivered air flows past the pressure sensor 54 and the flow sensor 53 before being supplied to the evaporator 1. Behind evaporator 1 the air is supplied to the patient 46 via respiratory tube 43 and face mask 44. Valve 55 is opened during the expiration phases so as to get rid of the exhaled air via respiratory tube 45.

The controller 56 receives the signals from the pressure sensor 54 and the flow sensor 53 and determines therefrom inspiration and expiration phases. Based on this result valves 52 and 55, the speed of compressor 51 and evaporator 1, in particular its heating elements, are controlled. In one embodiment the evaporator 1 is heated merely during the inspiration phases, which is possible due to its small thermal inertia. Here, the number of heated heating elements may be chosen in response to the airflow measured by the flow sensor 53. The higher the airflow, the more heating elements may be heated. Here, one can orientate oneself by the mean airflow so that the number of heated heating elements remains constant during an inspiration phase. In another embodiment the number of heated heating elements may be matched to the measured airflow more promptly, so that fewer heating elements are heated at the beginning and end of an inspiration phase than in the middle of an inspiration phase

Also the frequency of the heart of individual heating elements may be chosen in response to the measured airflow. Here, particularly the velocity of the following flow of water may be taken into account, so that a heating element is heated again only after the thickness of the water film has reached an equilibrium thickness of at least 50%. Additionally or alternatively the heating power of the individual heating elements may be controlled by a pulse-width modulation, the frequency of which is far above the thermal time constant of a single heating element.

In controlling the evaporator 1 also the sensor signal of the temperature sensor 47 may be taken into account in order to prevent a condensation in the respiratory tube 43.

In another embodiment a humidity sensor 49 may be provided instead of or in addition to the temperature sensor 47, which measures the air humidity at one spot in the respiratory tube 43 between evaporator 1 and face mask 44. If both a temperature sensor 47 and a humidity sensor 49 are provided, a measured relative humidity may be converted to an absolute humidity and vice versa.

The humidity sensor 49 and the evaporator 1 may be connected to a control loop by the controller 56 so as to allow a very exact adjustment of the air humidity. This control takes advantageously place only during the inspiration, because this is the only time when an airflow from the respirator 41 to the patient 46 causes a short time delay between evaporator 1 and humidity sensor 49, thereby enabling a fast control. Moreover, the time constant of the control loop can be adjusted in response to the signal supplied by the flow sensor 53: The higher the flow, the faster the control.

In another embodiment the gas temperature and/or the air humidity in front of evaporator 1 can be measured, and the evaporator as well as the evaporated amount of liquid can be controlled correspondingly, so that the desired air humidity behind the evaporator is obtained. This may take place additionally or alternatively to a measurement by the sensors 47 and 49.

Though an evaporator according to the invention is, in most cases, operated as a humidifier, the scope of protection shall not be limited thereto because also a gas mixture having a partial pressure of oxygen which deviates from 210 mbar can be humidified, and/or, for example, essential oils may be used instead of water.

Instead of a respirator 41 for intensive care medicine applications an evaporator according to the invention may also advantageously be employed together with CPAP- or bi-level apparatus due to its small power consumption and its small thermal inertia.

The invention was explained in more detail by means of preferred embodiments above. A person skilled in the art will appreciate, however, that various alterations and modifications may be made without departing from the spirit of the invention. Therefore, the scope of protection will be defined by the following claims and their equivalents.

### LIST OF REFERENCE NUMERALS

1 evaporator
2 storage tank assembly
3 bottom
4 inlet
5 outlet
6 tunnel
7 recess
8 resistive layer
9 upper metal strips
10 lower metal strips
11 contact surfaces
12 water film
13 heated heating element
14 steam drops
15 lime particles
16 recess
17 recess
18 contact surfaces
19 store room
20 humidifying area
41 respirator
43 respiratory tube (inspiration)
44 face mask
45 respiratory tube (expiration)
46 patient
47 temperature sensor
48 electric line
49 humidity sensor
51 compressor
52 valve
53 flow sensor
54 pressure sensor
55 expiration valve
56 controller

What is claimed is:

1. An evaporator, comprising:
a casing for receiving a liquid; and
a heater;
wherein
the heater comprises a plurality of heating elements which can be heated individually.

2. The evaporator according to claim 1, wherein the casing is formed such and the heater in the casing is arranged in an operating position such that a thin liquid film is formed on the heater.

3. The evaporator according to claim 1, wherein the casing has two connections, namely an inlet and an outlet, wherein the inlet serves to supply gas and the outlet serves to discharge gas, and wherein the discharged gas is accumulated with one of liquid molecules and atoms.

4. The evaporator according to claim 1, wherein the heater comprises a resistive layer contacted by one or more metal strips above and underneath thereof, wherein the upper and lower metal strips run approximately at right angles to each other when viewed from the normal to the resistive layer so that a heating element is formed in the overlapping region of an upper and a lower metal strip.

5. The evaporator according to claim 1, further comprising a controller and in that the casing is formed such and the heater in the casing is arranged in an operating position such that a thin liquid film is formed on the heater which is so thin that the controller can supply a specific heating element with such an amount of power that the liquid film above the specific heating element evaporates completely.

6. A respirator, comprising:
a compressor for delivering gas;
an evaporator comprising:
a casing for receiving a liquid; the casing having two connections, namely an inlet and an outlet, wherein the inlet serves to supply gas and the outlet serves to discharge gas, and wherein the discharged gas is accumulated with one of liquid molecules and atoms, the inlet of the casing being connected to the compressor for supplying gas by the connection; and
a heater comprising a plurality of heating elements which can be heated individually;
a respiratory tube, the end of which on the respirator's side being connected to the outlet of the casing; the respiratory tube comprising a patient's-side end;
a temperature sensor thermally contacting the respiratory tube; and
a controller, to which the signal of the temperature sensor is provided and which controls the temperature of the individual heating elements such that there is no water condensation in the respiratory tube.

7. The respirator according to claim 6, further comprising a flow sensor determining the flow of the delivered gas, wherein the signal of the flow sensor is supplied to the controller which determines inspiration and expiration phases from this signal and heats the heating elements only during an inspiration phase.

8. A respirator, comprising:
a compressor for delivering gas;
an evaporator comprising:
a casing for receiving a liquid; the casing having two connections, namely an inlet and an outlet, wherein the inlet serves to supply gas and the outlet serves to discharge gas, and wherein the discharged gas is accumulated with one of liquid molecules and atoms, the inlet of the casing being connected to the compressor for supplying gas by the connection; and
a heater comprising a plurality of heating elements which can be heated individually;
a flow sensor determining the flow of the delivered gas;
a controller, to which the signal of the flow sensor is provided, which determines inspiration and expiration phases from this signal and heats the heating elements only during an inspiration phase.

9. An evaporation method, comprising:
heating a liquid, wherein a portion of one of the molecules and atoms in the liquid evaporates;
heating a heating element of a heater which comprises a plurality of heating elements, wherein the liquid is in thermal contact with the heated heating element.

10. An evaporation method according to claim 9, further comprising:
forming a liquid film above the heater.

11. An evaporation method according to claim 9, further comprising:
supplying air through an inlet; and
discharging air accumulated with one of liquid molecules and atoms through an outlet.

12. An evaporation method according to claim 9, further comprising:
conducting an electric current through a resistive layer, wherein a voltage is applied between a metal strip above the resistive layer and a metal strip underneath the resistive layer, wherein the metal strips above the resistive layer and underneath the resistive layer run approximately at right angles to each other when viewed from the normal to the resistive layer.

13. An evaporation method according to claim 9, further comprising:
forming a liquid film above the heater; and
heating a heating element so that the liquid film above the heating element evaporates completely.

14. An evaporation method according to claim 9, further comprising:
measuring the temperature on a tube through which the gas accumulated with one of the liquid molecules and atoms is conducted; and
limiting the heating power of the heating element so that no liquid condenses in the tube.

15. An evaporation method according to claim 9, further comprising:
measuring the airflow through an evaporator;
determining inspiration and expiration phases from the airflow; and
heating heating elements only during the inspiration phases.
